SEQUENCE LISTING (6 sequences)

SEQ ID NO: 1 - protein, 37 aa, synthetic construct
VGRFRRLRKKTRKRLKKIGKVLKWIPPIVGSIPLGSG

SEQ ID NO: 2 - protein, 36 aa, synthetic construct
GRFRRLRKKTRKRLKKIGKVLKWIPPIVGSIPLGCG

SEQ ID NO: 3 - protein, 4 aa, synthetic construct
VGRF

SEQ ID NO: 4 - protein, 4 aa, synthetic construct
GRFR

SEQ ID NO: 5 - protein, 4 aa, synthetic construct
LGSG

SEQ ID NO: 6 - protein, 4 aa, synthetic construct
LGCG

(19) United States
(12) Patent Application Publication
(10) Pub. No.: US 2025/0002548 A1
(43) Pub. Date: Jan. 2, 2025

(54) Wild Boar Cathelicidin Peptide Variants and Vectors Encoding the Same for Uses in Managing Coronavirus Infections

(71) Applicants: Emory University, Atlanta, GA (US); Children's Healthcare of Atlanta, Inc., Atlanta, GA (US); Georgia Tech Research Corporation, Atlanta, GA (US)

(72) Inventors: Joshy Jacob, Atlanta, GA (US); Mehul Suthar, Atlanta, GA (US); Troy Von Beck, Atlanta, GA (US); Abigail Vanderheiden, Atlanta, GA (US); Nikita Mullick, Atlanta, GA (US); Jeffrey Skolnick, Atlanta, GA (US); Mu Gao, Atlanta, GA (US)

(21) Appl. No.: 18/708,508

(22) PCT Filed: Nov. 8, 2022
(86) PCT No.: PCT/US2022/079459

§ 371 Date: May 8, 2024

Related U.S. Application Data
(60) Provisional application No. 63/276,931, filed on Nov. 8, 2021.

(51) Int. Cl. C07K 14/47; A61K 38/00

(52) U.S. Cl. CPC C07K 14/4723; A61K 38/00

(57) ABSTRACT

This disclosure relates to wild boar cathelicidin peptides, fragments, variants, and derivatives thereof, and vectors encoding that same, as reported herein for uses in the prevention or treatment of viral infections such as coronavirus infections. In certain embodiments, the subject is a human subject exhibiting symptoms of, at risk of, or diagnosed with a coronaviral infection such as SARS-CoV2.

### CROSS-REFERENCE TO RELATED APPLICATIONS

This application claims the benefit of U.S. Provisional Application No. 63/276,931 filed Nov. 8, 2021. The entirety of this application is hereby incorporated by reference for all purposes.

### INCORPORATION-BY-REFERENCE OF MATERIAL SUBMITTED AS AN XML FILE VIA THE OFFICE ELECTRONIC FILING SYSTEM

The Sequence Listing associated with this application is provided in XML format and is hereby incorporated by reference into the specification. The name of the XML file containing the Sequence Listing is 21010PCT.xml. The XML file is 7 KB, was created on Nov. 7, 2022, and is being submitted electronically via the USPTO patent electronic filing system.

### BACKGROUND

Some common colds are due to certain coronavirus (CoV) strains associated with mild symptoms. More dangerous human strains such as severe acute respiratory syndrome associated coronavirus (SARS-CoV-1) and SARS-CoV-2 (also referred to as COVID-19) are believed to result from coronavirus strains jumping to humans by secondary zoonotic transfers. In humans, SARS-CoV-2 can be transferred from individuals who have mild symptoms or are asymptomatic and has caused numerous deaths worldwide. Thus, there is a need to identify improved therapies.

Storici et al. report an antibacterial peptide deduced from a pig myeloid cDNA. FEBS Lett, 1994, 337, (3), 303-307. See also WO2017091734.

Elnagdy et al. report the potential of using antimicrobial peptides as an antiviral therapy against COVID-19. ACS Pharmacol Transl Sci, 2020, 3, 780-782.

Lame et al. report ACE2-derived peptides inhibit SARS-CoV-2. Bioconjugate Chem, 2021, 32, 215-223.

References cited herein are not an admission of prior art.

### SUMMARY

This disclosure relates to method of managing coronavirus infections using wild boar cathelicidin peptide, variants, and vectors encoding the same. In certain embodiments, this disclosure relates to methods of treating or preventing a viral infection comprising administering an effective amount of a peptide, or a vector encoding a peptide, disclosed herein. In certain embodiments, the subject is a human subject exhibiting symptoms of, at risk of, or diagnosed with a coronaviral infection such as SARS-CoV2.

In certain embodiments, the peptide comprises or consists of the amino acid sequence of VGRFRRLRKKTRKRLKKIGKVLKWIPPIVGSIPLGSG (SEQ ID NO: 1), fragments, or variants thereof. In certain embodiments, the peptide comprises or consists of the amino acid sequence of GRFRRLRKKTRKRLKKIGKVLKWIPPIVGSIPLGCG (SEQ ID NO: 2), fragments, or variants thereof. In certain embodiments, the peptide has a N-terminus comprising or consisting of the amino acid sequence VGRF (SEQ ID NO: 3) or GRFR (SEQ ID NO: 4). In certain embodiments, the peptide has a C-terminus comprising or consisting of the amino acid sequence of LGSG (SEQ ID NO: 5) or LGCG (SEQ ID NO: 6).

In certain embodiments, the coronavirus infection is severe acute respiratory syndrome coronavirus 1 (SARS-CoV-1), SARS-CoV-2 (COVID-19), middle east respiratory syndrome coronavirus (MERS-CoV), HCoV-229E, HCoV-NL63, HCoV-OC43, or HCoV-HKU1.

In certain embodiments, the peptide, or vector encoding a peptide, is administered in combination with another antiviral agent.

In certain embodiments, the subject is more than 55, 65, or 75 years old.

In certain embodiments, the subject is diagnosed with a severe acute infection requiring intensive care.

In certain embodiments, a contemplated peptide with antiviral activity has the amino acid sequence VGRFRRLRKKTRKRLKKIGKVLKWIPPIVGSIPLGSG (SEQ ID NO: 1) or GRFRRLRKKTRKRLKKIGKVLKWIPPIVGSIPLGCG (SEQ ID NO: 2).

In certain embodiments, the variant peptide has at least one substitution, addition, or deletion such that the entire peptide is not naturally occurring.

In certain embodiments, this disclosure relates to recombinant vectors comprising a nucleic acid encoding a peptide disclosed herein and a heterologous nucleic acid sequence.

In certain embodiments, this disclosure relates to expression system comprising a vector encoding a peptide disclosed herein in operable combination with a heterologous promoter.

In certain embodiments, this disclosure relates to cells comprising a vector encoding a peptide disclosed herein.

In certain embodiments, this disclosure relates to pharmaceutical compositions comprising a peptide or vector disclosed herein and a pharmaceutically acceptable excipient.

In certain embodiments, this disclosure relates to the production of a medicament useful for treating or preventing a coronavirus infection or other viral infection.

### BRIEF DESCRIPTION OF THE SEVERAL VIEWS OF THE DRAWINGS

FIG. 1 shows data indicating a wild boar cathelicidin PMAP-36R inhibits SARS-CoV-2 infection of Vero E6 cells. PMAP-36R inhibits SARS-CoV-2 infection at lower concentrations than human LL-37.

FIG. 2 shows data indicating replacement of the PMAP-36R cysteine residue near the C-terminal end with serine (pSer) does not reduce SARS-CoV-2 inhibition.

FIG. 3 shows data indicating that the serine mutant (Yongshi) retains inhibitory activity against emergent SARS-CoV-2 variants alpha, beta, gamma, kappa, and delta.

### DETAILED DISCUSSION

Before the present disclosure is described in greater detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

An “embodiment” of this disclosure refers to an example and infers that the example is not necessarily limited to the example. Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

As used in this disclosure and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) have the meaning ascribed to them in U.S. Patent law in that they are inclusive or open-ended and do not exclude additional, unrecited elements or method steps.

“Consisting essentially of” or “consists of” or the like, when applied to methods and compositions encompassed by the present disclosure refers to the idea of excluding certain prior art element(s) as an inventive feature of a claim, but which may contain additional composition components or method steps, etc., that do not materially affect the basic and novel characteristic(s) of the compositions or methods, compared to those of the corresponding compositions or methods disclosed herein.

The term “comprising” in reference to a peptide having an amino acid sequence refers a peptide that may contain additional N-terminal (amine end) or C-terminal (carboxylic acid end) amino acids, i.e., the term is intended to include the amino acid sequence within a larger peptide. The term “consisting of” in reference to a peptide having an amino acid sequence refers a peptide having the exact number of amino acids in the sequence and not more or having not more than a rage of amino acids expressly specified in the claim. In certain embodiments, the disclosure contemplates that the “N-terminus of a peptide may consist of an amino acid sequence,” which refers to the N-terminus of the peptide having the exact number of amino acids in the sequence and not more or having not more than a rage of amino acids specified in the claim however the C-terminus may be connected to additional amino acids, e.g., as part of a larger peptide. Similarly, the disclosure contemplates that the “C-terminus of a peptide may consist of an amino acid sequence,” which refers to the C-terminus of the peptide having the exact number of amino acids in the sequence and not more or having not more than a rage of amino acids specified in the claim however the N-terminus may be connected to additional amino acids, e.g., as part of a larger peptide.

A “penultimate cysteine” refers to a cysteine amino acid being between the last amino acid on the C-terminus of the peptide and the rest of the peptide.

The terms “protein” and “peptide” refer to polymers comprising amino acids joined via peptide bonds and are used interchangeably. Amino acids may be naturally or non-naturally occurring. A “chimeric protein” or “fusion protein” is a molecule in which different portions of the protein are derived from different origins such that the entire molecule is not naturally occurring. A chimeric protein may contain amino acid sequences from the same species or different species as long as they are not arranged together in the same way that they exist in a natural state. Examples of a chimeric protein include sequences disclosed herein that contain one, two or more amino acids attached to the C-terminal or N-terminal end that are not identical to any naturally occurring protein, such as in the case of adding an amino acid containing an amine side chain group, e.g., lysine, an amino acid containing a carboxylic acid side chain group such as aspartic acid or glutamic acid, a polyhistidine (HIS) tag, e.g., typically four or more histidine amino acids, a human influenza hemagglutinin (HA) tag, a TAT polypeptide, GST peptide, or a FLAG tag.

A “variant” refers to a chemically similar sequence because of amino acid changes or chemical derivative thereof. In certain embodiments, a variant contains one or two, or more amino acid deletions or substitutions. In certain embodiments, the substitutions are conserved substitutions. In certain embodiments, a variant contains one, two, or ten or more, or ten or less amino acid additions. In certain embodiments, the additions may be to the N-terminus or the C-terminus. The variant may be substituted with one or more chemical substituents.

One type of conservative amino acid substitutions refers to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. A variant may have “non-conservative” changes (e.g., replacement of a glycine with a tryptophan). Similar minor variations may also include amino acid deletions or insertions (in other words, additions), or both. Guidance in determining which and how many amino acid residues may be substituted, inserted, or deleted without abolishing biological activity may be found using computer programs well known in the art. Variants can be tested in functional assays. Certain variants have less than 10%, and preferably less than 5%, and still more preferably less than 2% changes (whether substitutions, deletions, and so on). Variants can be prepared for testing by mutating a vector to produce appropriate codon alternatives for peptide translation.

As used herein, the term “derivative” refers to a structurally similar peptide that retains sufficient functional attributes of the identified analogue. The derivative may be structurally similar because it is lacking one or more atoms, e.g., replacing an amino group, hydroxyl, or thiol group with a hydrogen, substituted, a salt, in different hydration/oxidation states, or because one or more atoms within the molecule are switched, such as, but not limited to, replacing an oxygen atom with a sulfur atom, or replacing an amino group with a hydroxyl group. The derivative may be a prodrug, comprise a lipid, polyethylene glycol, saccharide, polysaccharide. A derivative may be two or more peptides linked together by a linking group. It is contemplated that the linking group may be biodegradable. Derivatives may be prepared by any variety of synthetic methods or appropriate adaptations presented in synthetic or organic chemistry textbooks, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

In certain embodiments, the peptides disclosed herein have at least one non-naturally occurring molecular modification, such as the attachment of polyethylene glycol, the attachment of a chimeric peptide, the attachment of a fluorescent dye comprising aromatic groups, fluorescent peptide, a chelating agent capable of binding a radionuclide, or ¹⁸F. In certain embodiments, the peptides contain an N-terminal acetyl, propionyl group, myristoyl and palmitoyl, group or N-terminal mono- or di-methylation, or a C-terminal alkyl ester or amide. In certain embodiments, this disclosure contemplates peptides disclosed herein labeled using commercially available biotinylation reagents. Biotinylated peptide can be used in streptavidin/avidin affinity binding, purification, and detection. In certain embodiments, the disclosure contemplates a peptide disclose herein containing azide-derivatives of naturally occurring monosaccharides such as N-azidoacetylglucosamine, N-azidoacetylmannosamine, and N-azidoacetylgalactosamine.

In certain embodiments, this disclosure contemplates derivatives of peptide disclose herein wherein one or more amino acids are substituted with chemical groups to improve pharmacokinetic properties such as solubility and serum half-life, optionally connected through a linker. In certain embodiments, such a derivative may be a prodrug wherein the substituent or linker is biodegradable, or the substituent or linker is not biodegradable. In certain embodiments, contemplated substituents include a saccharide, polysaccharide, acetyl, fatty acid, lipid, and/or polyethylene glycol. The substituent may be covalently bonded through the formation of amide bonds on the C-terminus or N-terminus of the peptide optionally connected through a linker. In certain embodiments, it is contemplated that the substituent may be covalently bonded through an amino acid within the peptide, e.g., through an amine side chain group such as lysine or an amino acid containing a carboxylic acid side chain group such as aspartic acid or glutamic acid, within the peptide comprising a sequence disclosed herein. In certain embodiments, it is contemplated that the substituent may be covalently bonded through a cysteine in a sequence disclosed herein optionally connected through a linker. In certain embodiments, a substituent is connected through a linker that forms a disulfide with a cysteine amino acid side group.

The term “substituted” refers to a molecule wherein at least one hydrogen atom is replaced with a substituent. When substituted, one or more of the groups are “substituents.” The molecule may be multiply substituted. In the case of an oxo substituent (“═O”), two hydrogen atoms are replaced. Example substituents within this context may include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRaRb, —NRaC(═O)Rb, —NRaC(═O)NRaNRb, —NRaC(═O)ORb, —NRaSO₂Rb, —C(═O)Ra, —C(═O)ORa, —C(═O)NRaRb, —OC(═O)NRaRb, —ORa, —SRa, —SORa, —S(═O)₂Ra, —OS(═O)₂Ra and —S(═O)₂ORa. Ra and Rb in this context may be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, and heteroarylalkyl. The substituents may further optionally be substituted.

As used herein, a “lipid” group refers to a hydrophobic group that is naturally or non-naturally occurring that is highly insoluble in water. As used herein a lipid group is considered highly insoluble in water when the point of connection on the lipid is replaced with a hydrogen and the resulting compound has a solubility of less than 0.63×10⁻⁴ % w/w (at 25° C.) in water, which is the percent solubility of octane in water by weight. See Solvent Recovery Handbook, 2^{nd} Ed, Smallwood, 2002 by Blackwell Science, page 195. Examples of naturally occurring lipids include saturated or unsaturated hydrocarbon chains found in fatty acids, glycerolipids, cholesterol, steroids, polyketides, and derivatives. Non-naturally occurring lipids include derivatives of naturally occurring lipids, acrylic polymers, aromatic, and alkylated compounds and derivatives thereof.

The term “prodrug” refers to an agent that is converted into a biologically active form in vivo. Prodrugs are often useful because, in some situations, they may be easier to administer than the parent compound. The prodrug may also have improved solubility in pharmaceutical compositions over the parent drug. A prodrug may be converted into the parent drug by various mechanisms, including enzymatic processes and metabolic hydrolysis. Typical prodrugs are pharmaceutically acceptable esters. Prodrugs include compounds wherein a hydroxy, amino or mercapto (thiol) group is bonded to any group that, when the prodrug of the active compound is administered to a subject, cleaves to form a free hydroxy, free amino or free mercapto group, respectively. Examples of prodrugs include, but are not limited to, acetate, formate, and benzoate derivatives of an alcohol or acetamide, formamide and benzamide derivatives of an amine functional group in the active compound and the like.

As used herein, “pharmaceutically acceptable esters” include, but are not limited to, alkyl, alkenyl, alkynyl, aryl, arylalkyl, and cycloalkyl esters of acidic groups, including, but not limited to, carboxylic acids, phosphoric acids, phosphinic acids, sulfonic acids, sulfinic acids, and boronic acids.

As used herein, the term “conjugated” refers to linking molecular entities through covalent bonds, linking groups, or by other specific binding interactions, such as due to hydrogen bonding or other van der Walls forces. The force to break a covalent bond is high, e.g., about 1500 pN for a carbon-to-carbon bond. The force to break a combination of strong protein interactions is typically a magnitude less, e.g., biotin to streptavidin is about 150 pN. Thus, a skilled artisan would understand that conjugation must be strong enough to bind molecular entities in order to implement the intended results.

A “linking group” refers to any variety of molecular arrangements that can be used to bridge two molecular moieties together. An example formula may be —Rₘ— wherein R is selected individually and independently at each occurrence as: —CRₘRₘ—, —CHRₘ—, —CH—, —C—, —CH₂—, —C(OH)Rₘ, —C(OH)(OH)—, —C(OH)H, —C(Hal)Rₘ—, —C(Hal)(Hal)—, —C(Hal)H—, —C(N₃)Rₘ—, —C(CN)Rₘ—, —C(CN)(CN)—, —C(CN)H—, —C(N₃)(N₃)—, —C(N₃)H—, —O—, —S—, —N—, —NH—, —NRₘ—, —(C═O)—, —(C═NH)—, —(C═S)—, —(C═CH₂)—, which may contain single, double, or triple bonds individually and independently between the R groups. If an R is branched with an Rₘ it may be terminated with a group such as —CH₃, —H, —CH═CH₂, —CCH, —OH, —SH, —NH₂, —N₃, —CN, or —Hal, or two branched Rs may form a cyclic structure. It is contemplated that in certain instances, the total Rs or “m” may be less than 100, or 50, or 25, or 10. Examples of linking groups include bridging alkyl groups and alkoxyalkyl groups. Linking groups may be substituted with one or more sub stituents.

As used herein, the term “biodegradable” in reference to a substituent or linker refers to a molecular arrangement in a peptide derivative that when administered to a subject, e.g., human, will be broken down by biological mechanism such that a metabolite will be formed and the molecular arrangement will not persist for over a long period of time, e.g., the molecular arrangement will be broken down by the body after a several hours or days. In certain embodiments, the disclosure contemplates that the biodegradable linker or substituent will not exist after a week or a month.

As used herein, the term “sterilized” refers to subjecting something to a process that effectively kills or eliminates transmissible agents (such as fungi, bacteria, viruses, prions, and spore forms etc.). Sterilization can be achieved through application of heat, chemicals, irradiation, high pressure, or filtration. One process involves water prepared by distillation and stored in an airtight container wherein suitable additives are introduced to approximate isotonicity.

“Subject” refers to any animal, preferably a human patient, livestock, rodent, monkey, or domestic pet.

As used herein, the terms “prevent” and “preventing” include the prevention of the recurrence, spread or onset. It is not intended that the present disclosure be limited to complete prevention. In some embodiments, the onset is delayed, or the severity of the disease is reduced.

As used herein, the terms “treat” and “treating” are not limited to the case where the subject (e.g., patient) is cured and the disease is eradicated. Rather, embodiments, of the present disclosure also contemplate treatment that merely reduces symptoms, and/or delays disease progression.

As used herein, the term “combination with” when used to describe administration with an additional treatment means that the agent may be administered prior to, together with, or after the additional treatment, or a combination thereof.

The term “effective amount” refers to that amount of a compound or pharmaceutical composition described herein that is sufficient to effect the intended application including, but not limited to, disease treatment, as illustrated below. The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The specific dose will vary depending on, for example, the particular compounds chosen, the dosing regimen to be followed, whether it is administered in combination with other agents, timing of administration, the tissue to which it is administered, and the physical delivery system in which it is carried.

The term “nucleic acid” refers to a polymer of nucleotides, or a polynucleotide, e.g., RNA, DNA, or a combination thereof. The term is used to designate a single molecule, or a collection of molecules. Nucleic acids may be single stranded or double stranded and may include coding regions and regions of various control elements.

A “heterologous” nucleic acid sequence or peptide sequence refers to a nucleic acid sequence or peptide sequence that do not naturally occur, e.g., because the whole sequences contain a segment from other plants, bacteria, viruses, other organisms, or joinder of two sequences that occur the same organism but are joined together in a manner that does not naturally occur in the same organism or any natural state.

The term “recombinant” when made in reference to a nucleic acid molecule refers to a nucleic acid molecule which is comprised of segments of nucleic acid joined together by means of molecular biological techniques provided that the entire nucleic acid sequence does not occurring in nature, i.e., there is at least one mutation in the overall sequence such that the entire sequence is not naturally occurring even though separately segments may occur in nature. The segments may be joined in an altered arrangement such that the entire nucleic acid sequence from start to finish does not naturally occur. The term “recombinant” when made in reference to a protein or a peptide refers to a protein molecule that is expressed using a recombinant nucleic acid molecule.

The terms “vector” or “expression vector” refer to a recombinant nucleic acid containing a desired coding sequence and appropriate nucleic acid sequences necessary for the expression of the operably linked coding sequence in a particular host organism or expression system, e.g., cellular or cell-free expression system. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals. In certain embodiments, this disclosure contemplates a vector encoding a peptide disclosed herein in operable combination with a heterologous promoter.

Protein “expression systems” refer to in vivo and in vitro (cell free) systems. Systems for recombinant protein expression typically utilize somatic cells transfected with a DNA expression vector that contains the template. The cells are cultured under conditions such that they translate the desired protein. Expressed proteins are extracted for subsequent purification. In vivo protein expression systems using prokaryotic and eukaryotic cells are well known. Proteins may be recovered using denaturants and protein-refolding procedures. In vitro (cell-free) protein expression systems typically use translation-compatible extracts of whole cells or compositions that contain components sufficient for transcription, translation, and optionally post-translational modifications such as RNA polymerase, regulatory protein factors, transcription factors, ribosomes, tRNA cofactors, amino acids, and nucleotides. In the presence of an expression vectors, these extracts and components can synthesize proteins of interest. Cell-free systems typically do not contain proteases and enable labeling of the protein with modified amino acids. See, e.g., Shimizu et al., Cell-free translation reconstituted with purified components, 2001, Nat. Biotechnol., 19, 751-755 and Asahara & Chong, Nucleic Acids Research, 2010, 38 (13):e141, both hereby incorporated by reference in their entirety.

A “selectable marker” is a nucleic acid introduced into a recombinant vector that encodes a peptide that confers a trait suitable for artificial selection or identification (report gene), e.g., beta-lactamase confers antibiotic resistance, which allows an organism expressing beta-lactamase to survive in the presence antibiotic in a growth medium. Another example is thymidine kinase, which makes the host sensitive to ganciclovir selection. It may be a screenable marker that allows one to distinguish between wanted and unwanted cells based on the presence or absence of an expected color. For example, the lac-z-gene produces a beta-galactosidase enzyme that confers a blue color in the presence of X-gal (5-bromo-4-chloro-3-indolyl-β-D-galactoside). If recombinant insertion inactivates the lac-z-gene, then the resulting colonies are colorless. There may be one or more selectable markers, e.g., an enzyme that can complement to the inability of an expression organism to synthesize a particular compound required for its growth (auxotrophic) and one able to convert a compound to another that is toxic for growth. Additional contemplated selectable markers include any genes that impart antibacterial resistance or express a fluorescent protein. Examples include, but are not limited to, the following genes: amp^{r}, cam^{r}, tet^{r}, blasticidin^{r}, neo^{r}, hyg^{r}, abx^{r}, neomycin phosphotransferase type II gene (nptII), p-glucuronidase (gus), green fluorescent protein (gfp), egfp, yfp, mCherry, p-galactosidase (lacZ), lacZa, lacZAM15, chloramphenicol acetyltransferase (cat), alkaline phosphatase (phoA), bacterial luciferase (luxAB), bialaphos resistance gene (bar), phosphomannose isomerase (pmi), xylose isomerase (xylA), arabitol dehydrogenase (atlD), UDP-glucose: galactose-1-phosphate uridyltransferasel (galT), feedback-insensitive α subunit of anthranilate synthase (OASA1D), 2-deoxyglucose (2-DOGR), benzyladenine-N-3-glucuronide, E. coli threonine deaminase, glutamate 1-semialdehyde aminotransferase (GSA-AT), D-amino acidoxidase (DAAO), salt-tolerance gene (rstB), ferredoxin-like protein (pflp), trehalose-6-P synthase gene (AtTPS1), lysine racemase (lyr), dihydrodipicolinate synthase (dapA), tryptophan synthase beta 1 (AtTSB1), dehalogenase (dhlA), mannose-6-phosphate reductase gene (M6PR), hygromycin phosphotransferase (HPT), and D-serine ammonialyase (dsdA).

A “label” refers to a detectable moiety that is conjugated directly or indirectly to another molecule, such as an antibody or a protein, to facilitate detection of that molecule. Specific, non-limiting examples of labels include fluorescent tags, enzymatic linkages, and radioactive isotopes. In one example, a “label receptor” refers to incorporation of a heterologous peptide in the receptor. A label includes the incorporation of a radiolabeled amino acid or the covalent attachment of biotinyl moieties to a peptide that can be detected by marked avidin (for example, streptavidin containing a fluorescent marker or enzymatic activity that can be detected by optical or colorimetric methods). Various methods of labeling peptides and glycoproteins are known in the art and may be used. Examples of labels for peptides include, but are not limited to, the following: radioisotopes or radionucleotides (such as ³⁵S or ¹³¹I), fluorescent labels (such as fluorescein isothiocyanate (FITC), rhodamine, lanthanide phosphors), enzymatic labels (such as horseradish peroxidase, beta-galactosidase, luciferase, alkaline phosphatase), chemiluminescent markers, biotinyl groups, predetermined peptide epitopes recognized by a secondary reporter (such as a leucine zipper pair sequences, binding sites for secondary antibodies, metal binding domains, epitope tags), or magnetic agents, such as gadolinium chelates. In some embodiments, labels are attached by spacer arms (linking groups) of various lengths to reduce potential steric hindrance.

In certain embodiments, the disclosure relates to recombinant peptides comprising sequences disclosed herein or variants or fusions thereof wherein the amino terminal end or the carbon terminal end of the amino acid sequence are optionally attached to a heterologous amino acid sequence, label, or reporter molecule.

In certain embodiments, the disclosure relates to the recombinant vectors comprising a nucleic acid encoding a peptide disclosed herein or chimeric protein thereof.

In certain embodiments, the recombinant vector optionally comprises a mammalian, human, insect, viral, bacterial, bacterial plasmid, yeast associated origin of replication or gene such as a gene or retroviral gene or lentiviral LTR, TAR, RRE, PE, SLIP, CRS, and INS nucleotide segment or gene selected from tat, rev, nef, vif, vpr, vpu, and vpx or structural genes selected from gag, pol, and env.

In certain embodiments, the recombinant vector optionally comprises a gene vector element (nucleic acid) such as a selectable marker region, lac operon, a CMV promoter, a hybrid chicken B-actin/CMV enhancer (CAG) promoter, tac promoter, T7 RNA polymerase promoter, SP6 RNA polymerase promoter, SV40 promoter, internal ribosome entry site (IRES) sequence, cis-acting woodchuck post regulatory element (WPRE), scaffold-attachment region (SAR), inverted terminal repeats (ITR), FLAG™ tag coding region, c-myc tag coding region, metal affinity tag coding region, streptavidin binding peptide tag coding region, polyHis tag coding region, HA tag coding region, MBP tag coding region, GST tag coding region, polyadenylation coding region, SV40 polyadenylation signal, SV40 origin of replication, Col E1 origin of replication, f1 origin, pBR322 origin, or pUC origin, TEV protease recognition site, loxP site, Cre recombinase coding region, or a multiple cloning site such as having 5, 6, or 7 or more restriction sites within a continuous segment of less than 50 or 60 nucleotides or having 3 or 4 or more restriction sites with a continuous segment of less than 20 or 30 nucleotides.

Sequence “identity” refers to the number of exactly matching amino acids (expressed as a percentage) in a sequence alignment between two sequences of the alignment calculated using the number of identical positions divided by the greater of the shortest sequence or the number of equivalent positions excluding overhangs wherein internal gaps are counted as an equivalent position. In certain embodiments, any recitation of sequence identity expressed herein may be substituted for sequence similarity. Percent “similarity” is used to quantify the similarity between two sequences of the alignment. This method is identical to determining the identity except that certain amino acids do not have to be identical to have a match. Amino acids are classified as matches if they are among a group with similar properties according to the following amino acid groups: Aromatic—F Y W; hydrophobic—A V I L; Charged positive: R K H; Charged negative—D E; Polar—S T N Q.

### Antiviral Peptides

In certain embodiments, the peptides of this disclosure are wild boar cathelicidin peptides, variants, derivatives, and prodrugs thereof. In certain embodiments, the peptides of this disclosure comprise or consist of the amino acid sequences of SEQ ID NO: 1 or 2, fragments, derivatives, or variants thereof. In certain embodiments, the peptide comprises or consists of the amino acid sequence of VGRFRRLRKKTRKRLKKIGKVLKWIPPIVGSIPLGSG (SEQ ID NO: 1), derivatives, fragments, or variants thereof. In certain embodiments, the peptide comprises or consists of the amino acid sequence of GRFRRLRKKTRKRLKKIGKVLKWIPPIVGSIPLGCG (SEQ ID NO: 2), derivatives, fragments, or variants thereof. In certain embodiments, the peptide has a N-terminus comprising or consisting of the amino acid sequence of VGRF (SEQ ID NO: 3) or GRFR (SEQ ID NO: 4). In certain embodiments, the peptide has a C-terminus comprising or consisting of the amino acid sequence LGSG (SEQ ID NO: 5) or LGCG (SEQ ID NO: 6).

In certain embodiments, the disclosure contemplates peptides disclosed herein having at least one molecular modification, e.g., such that the peptide contains a non-naturally occurring amino acid or amino acid sequence. In certain embodiments, the disclosure contemplates a non-naturally occurring derivative of a peptide having SEQ ID NO: 1 or 2, fragments, derivatives, or variants thereof. In certain embodiments, the disclosure contemplates a derivative in the form of a prodrug. In certain embodiments, the disclosure contemplates a derivative wherein an amino, carboxyl, hydroxyl, or thiol group in a peptide disclosed herein is substituted. In certain embodiments, the disclosure contemplates peptides disclosed herein having a label, e.g., fluorescent label or radioactive label.

In certain embodiments, the peptide comprises one or more of the following modifications: N-terminal acetylation, C-terminal amidation, PEGylation, sulfation, addition of hydroxyl group to one or more proline residues.

In certain embodiments, this disclosure relates to fusion proteins comprising peptides disclosed, e.g., a fusion with an immunoglobulin constant region (Fc) or albumin.

In certain embodiments, the disclosure contemplates a peptide having greater than 50%, 60%, 70%, 80%, 90%, 95% sequence identity or similarity to SEQ ID NO: 1 or 2 and contains at least one substitution and/or modification relative to SEQ ID NO: 1 or 2 such that the entire peptide is not naturally occurring, e.g., one or more amino acids have been changed relative to any natural sequence.

In certain embodiments, the variants have one, two, three, or more amino acid substitutions, insertions, or deletions. In certain embodiments, the peptides are produced synthetically or recombinantly. In certain embodiments, the peptides have at least one non-naturally occurring amino acid substitution, addition, or deletion. In certain embodiments, the amino acid substitutions are conserved substitutions.

In certain embodiments, the disclosure relates to recombinant vectors comprising a nucleic acid encoding peptide disclosed herein. In certain embodiments, the disclosure relates to expression systems comprising a vector comprising a nucleic acid encoding peptide disclosed herein. In certain embodiments, the disclosure relates to cells comprising a vector comprising a nucleic acid encoding peptide disclosed herein. In certain embodiments, the disclosure relates to a vector comprising the nucleic acid encoding a peptide disclosed herein and a heterologous nucleic acid sequence. In certain embodiments, the disclosure relates to a vector comprising the nucleic acid encoding a peptide disclosed herein in operable combination with a heterologous promoter sequence.

### Methods of Use

This disclosure relates to method of managing viral infections using a peptide, or vector encoding a peptide, disclosed herein and derivatives and variants disclosed herein. In certain embodiments, this disclosure relates to methods of treating or preventing a viral infection comprising administering an effective amount of a peptide, or a vector encoding a peptide, disclosed herein. In certain embodiments, the subject is exhibiting symptoms of, at risk of, or diagnosed with a coronaviral infection such as SARS-CoV2.

In certain embodiments, the peptide comprises or consists of the amino acid sequence of VGRFRRLRKKTRKRLKKIGKVLKWIPPIVGSIPLGSG (SEQ ID NO: 1) or the amino acid sequence of VGRFRRLRKKTRKRLKKIGKVLKWIPPIVGSIPLGCG (SEQ ID NO: 2), fragments, derivatives, or variants thereof.

In certain embodiments, the viral infection is a coronavirus infection such as a coronavirus, severe acute respiratory syndrome coronavirus 1 (SARS-CoV-1), SARS-CoV-2 (COVID-19), middle east respiratory syndrome coronavirus (MERS-CoV), HCoV-229E, HCoV-NL63, HCoV-OC43, or HCoV-HKU1.

In certain embodiments, the subject is more than 55, 65, or 75 years old.

In certain embodiments, the subject is diagnosed with a severe acute infection requiring intensive care.

In certain embodiments, the subject is diagnosed with a severe acute infection requiring intensive care. In certain embodiments, the subject is any age, e.g., less than 25, 20, 15, or 10 years old or the subject is more than 55, 65, or 75 years old.

In certain embodiments, this disclosure relates to treating or preventing chronic acute respiratory syndrome or associated side effects due to a coronavirus or other viral infections comprising administering a composition comprising an effective amount of a peptide, or vector encoding a peptide, disclosed herein to a subject in need thereof. In certain embodiments, the subject is diagnosed with a viral infection that poses a risk of developing an acute respiratory syndrome or chronic acute respiratory syndrome such as subject diagnosed with a high-risk coronavirus infection, e.g., SARS-CoV-1 or SARS-CoV-2 infection.

In certain embodiments, the peptide, or vector encoding a peptide, is administered daily. In certain embodiments, the peptide, or vector encoding a peptide, is administered daily for more than 3, 5, 7 days or two weeks.

In certain embodiments, the peptide, or vector encoding a peptide, is administered daily, up to 2 times a day, 3 times a day, or as a continuous infusion. In certain embodiments, the peptide, or vector encoding a peptide, is administered as a continuous enteral feeding for severely ill and/or hospitalized subjects.

In certain embodiments, the subject is diagnosed with fatigue, shortness of breath, anxiety, depression, brain fog, joint pain, and/or chest pain, and optionally diabetes, stroke, heart rhythm abnormality, and/or blood clot in the lungs.

In certain embodiments, the subject is more than 55, 65, or 75 years old and/or diagnosed with a severe acute infection requiring intensive care, pre-existing respiratory illness, obesity, diabetes, high blood pressure, chronic cardiovascular disease, chronic kidney disease, organ transplant, or cancer.

In certain embodiments, the peptide, or vector encoding a peptide, disclosed herein is administered to a subject that cohabitates with a person diagnosed with a coronaviral infection in order to prevent spread of the infection.

In certain embodiments, the peptide, or vector encoding a peptide, is administered in combination with another antiviral agent such as remdesivir, chloroquine, hydroxychloroquine, azithromycin, ivermectin, lopinavir, ritonavir, nitazoxanide, or combinations thereof.

In certain embodiments, the peptide, or vector encoding a peptide, disclosed herein is administered in combination with another anti-viral agent such as, baricitinib, liraglutide, molnupiravir, salinomycin, 1-(4-chlorophenyl)-N-{3-cyano-4-[4-(morpholin-4-yl)piperidin-1-yl]phenyl}-5-methyl-1H-pyrazole-4-carboxamide (Y-320), (5-(2,4-bis(3-methylmorpholino)pyrido[2,3-d]pyrimidin-7-yl)-2-methoxyphenyl)methanol (AZD8055), bemcentinib, dacomitinib, 1-(4-(4-(8-oxa-3-azabicyclo[3.2.1]octan-3-yl)-1-(1,4-dioxaspiro[4.5]decan-8-yl)-1H-pyrazolo[3,4-d]pyrimidin-6- yl)phenyl)-3-methylurea (WYE-125132), ebastine, cyclosporine, fenofibrate, fenofibric acid, masitinib, proguanil, sulfasalazine, specific binding agents that bind the receptor binding domain of the SARS-CoV-2 spike protein S1 subunit (residues V16-R685; GenBank: QHD43416), monoclonal antibodies, casirivimab, imdevimab bamlanivimab, etesevimab, tixagevimab (AZD8895), cilgavimab, and combinations thereof.

In certain embodiments, the peptide, or vector encoding a peptide, disclosed herein is administered in combination with heparin.

In certain embodiments, the peptide, or vector encoding a peptide, disclosed herein is administered in combination with anti-inflammatory agents such as dexamethasone or baricitinib.

In certain embodiments, the peptide, or vector encoding a peptide, disclosed herein is administered in combination with pentoxifylline.

Although embodiments of this disclosure contemplate treatment of coronavirus infections, management of other viral infections are contemplated such as influenza virus, rhinovirus, hepatitis A, hepatitis B, hepatitis C, human papillomaviruses, human immunodeficiency, herpes virus, Epstein-Barr virus, herpes simplex virus, varicella-zoster virus, shingles virus, mumps virus, measles virus, West Nile virus, poliovirus, non-poliovirus enterovirus, respiratory syncytial virus, and parainfluenza virus.

In certain embodiments, the peptide, or vector encoding a peptide, disclosed herein is administered in combination with another or second therapeutic agent or antiviral agent. In certain embodiments, the antiviral agent(s) is abacavir, acyclovir, acyclovir, adefovir, amantadine, amprenavir, ampligen, arbidol, atazanavir, atripla, boceprevir, cidofovir, combivir, darunavir, delavirdine, didanosine, docosanol, dolutegravir, edoxudine, efavirenz, emtricitabine, enfuvirtide, entecavir, famciclovir, fomivirsen, fosamprenavir, foscarnet, fosfonet, ganciclovir, ibacitabine, imunovir, idoxuridine, imiquimod, indinavir, inosine, interferon type III, interferon type II, interferon type I, lamivudine, lopinavir, loviride, maraviroc, moroxydine, methisazone, nelfinavir, nevirapine, nexavir, oseltamivir, peginterferon alfa-2a, penciclovir, peramivir, pleconaril, podophyllotoxin, raltegravir, ribavirin, rimantadine, ritonavir, saquinavir, stavudine, tenofovir, tenofovir disoproxil, tenofovir alafenamide fumarate, tipranavir, trifluridine, trizivir, tromantadine, truvada, valaciclovir, valganciclovir, vicriviroc, vidarabine, viramidine, zalcitabine, zanamivir, zidovudine, and combinations thereof.

Methods of administering peptides include, but are not limited to, parenteral administration (e.g., intradermal, intramuscular, intraperitoneal, intravenous, and subcutaneous), epidural, and mucosal (e.g., intranasal and oral routes). In certain embodiment, the peptides or chimeric proteins are administered intramuscularly, intravenously, or subcutaneously. The compositions may be administered by any convenient route, for example, by infusion or bolus injection, by absorption through epithelial or mucocutaneous linings (e.g., oral mucosa, rectal and intestinal mucosa, etc.) and may be administered together with other biologically active agents. Administration can be systemic or local. In addition, pulmonary administration can also be employed, e.g., by use of an inhaler or nebulizer, and formulation with an aerosolizing agent.

The precise dose to be employed in the formulation will also depend on the route of administration, and the seriousness of the condition, and should be decided according to the judgment of the practitioner and each patient's circumstances. Effective doses may be extrapolated from dose-response curves derived from in vitro or animal model test systems. For peptides and fusion proteins, the dosage administered to a patient is typically 0.0001 mg/kg to 100 mg/kg of the patient's body weight. Preferably, the dosage administered to a patient is between 0.0001 mg/kg and 20 mg/kg, 0.0001 mg/kg and 10 mg/kg, 0.0001 mg/kg and 5 mg/kg, 0.0001 and 2 mg/kg, 0.0001 and 1 mg/kg, 0.0001 mg/kg and 0.75 mg/kg, 0.0001 mg/kg and 0.5 mg/kg, 0.0001 mg/kg to 0.25 mg/kg, 0.0001 to 0.15 mg/kg, 0.0001 to 0.10 mg/kg, 0.001 to 0.5 mg/kg, 0.01 to 0.25 mg/kg or 0.01 to 0.10 mg/kg of the patient's body weight. Further, the dosage and frequency of administration of proteins may be reduced by enhancing uptake and tissue penetration of the fusion proteins by modifications such as, for example, lipidation.

In certain embodiments, this disclosure contemplates the production of a medicament comprising peptides disclosed herein and uses for methods disclosed herein.

### Pharmaceutical Compositions

In certain embodiments, this disclosure relates to compositions comprising a peptide disclosed herein. In certain embodiments, the disclosure relates to pharmaceutical compositions comprising a peptide having SEQ ID NO: 1 or 2, fragments, variants, or derivatives thereof and a pharmaceutically acceptable excipient or carrier.

In certain embodiments, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete), excipient, or vehicle with which the therapeutic is administered. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. These compositions can take the form of solutions, suspensions, emulsion, tablets, pills, capsules, powders, gels, sustained-release formulations, and the like. Such pharmaceutical carriers can be sterile liquids, such as water and oils, including those of petroleum, animal, vegetable, or synthetic origin, such as peanut oil, soybean oil, mineral oil, sesame oil and the like.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents solvents or vehicles include water, ethanol, polyols (propylene glycol, polyethylene glycol, glycerol, and the like), suitable mixtures thereof, vegetable (such as olive oil, sesame oil) and injectable organic esters such as ethyl oleate.

In certain embodiments, the pharmaceutical composition is in the form of a sterilized pH buffered aqueous salt solution. In certain embodiments, the composition is an aqueous buffer, e.g., a pH between 6 and 8. Generally, the ingredients of compositions are supplied either separately or mixed together in unit dosage form, for example, as a dry lyophilized powder or water free concentrate in a hermetically sealed container such as an ampoule indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In certain embodiments, this disclosure relates to a pharmaceutical pack or kit comprising one or more containers, e.g., vial, filled with peptides disclosed herein. Optionally, one or more other prophylactic or therapeutic agents useful for the treatment of a disease can also be included in the pharmaceutical pack or kit.

One embodiment of this disclosure provides a pharmaceutical pack or kit including one or more containers filled with one or more of the ingredients of the pharmaceutical compositions. Optionally associated with such container(s) can be a notice in the form prescribed by a governmental agency regulating the manufacture, use or sale of pharmaceuticals or biological products, which notice reflects approval by the agency of manufacture, use or sale for human administration.

Components of the kit may be administration means, such as syringes, catheters, brushes, etc. (if the compositions are not already provided in the administration means) or other components necessary for use in medical (surgical) practice, such as substitute needles or catheters, extra vials or further wound cover means.

Providing a pharmaceutic composition is possible in a one-step process, simply by adding a suitable pharmaceutically acceptable diluent to the composition in a container. In certain embodiments, the container is preferably a syringe for administering the reconstituted pharmaceutical composition after contact with the diluent. In certain embodiments, the peptide can be filled into a syringe, and the syringe can then be closed with the stopper. A diluent is used in an amount to achieve the desired end-concentration. The pharmaceutical composition may contain other useful component, such as ions, buffers, excipients, stabilizers, etc.

A “dry” pharmaceutical composition typically has only a residual content of moisture, which may approximately correspond to the moisture content of comparable commercial products, for example, has about 12% moisture as a dry product. Usually, the dry pharmaceutical composition has a residual moisture content preferably below 10% moisture, more preferred below 5% moisture, especially below 1% moisture. The pharmaceutical composition can also have lower moisture content, e.g., 0.1% or even below. In certain embodiments, the pharmaceutical composition is provided in dry in order to prevent degradation and enable storage stability.

The pharmaceutical composition may then preferably be applied via specific needles of the syringe or via suitable catheters. A typical diluent comprises water for injection, and NaCl (preferably 50 to 150 mM, especially 110 mM), CaCl₂ (preferably 10 to 80 mM, especially 40 mM), sodium acetate (preferably 0 to 50 mM, especially 20 mM) and mannitol (preferably up to 10% w/w, especially 2% w/w). Preferably, the diluent can also include a buffer or buffer system so as to buffer the pH of the reconstituted dry composition, preferably at a pH of 6.2 to 7.5, especially at pH of 6.9 to 7.1.

In certain embodiments, the composition is in the form of a container configured to spray a liquid, or in the form of a sealed container with a propellant. In certain embodiments, the aerosolizing agent or propellant is a hydrofluoroalkane, 1,1,1,2-tetrafluoroethane, 1,1,1,2,3,3,3-heptafluoropropane, propane, n-butane, isobutene, carbon dioxide, air, nitrogen, nitrous oxide, dimethyl ether, trans-1,3,3,3-tetrafluoroprop-1-ene, or combinations thereof.

Solid dosage forms for oral administration include capsules, tablets, pills, powders, and granules. In such solid dosage forms, the proteins may be admixed with at least one inert customary excipient (or carrier) such as sodium citrate or dicalcium phosphate or: (a) fillers or extenders, as for example, starches, lactose, sucrose, glucose, mannitol and silicic acid, (b) binders, as for example, carboxymethylcellulose, alginates, gelatin, polyvinylpyrrolidone, sucrose, and acacia, (c) humectants, as for example, glycerol (d) disintegrating agents, as for example, agar-agar, calcium carbonate, potato or tapioca starch, alginic acid, certain complex silicates, and sodium carbonate, (e) solution retarders, as for example paraffin, (f) absorption accelerators, as for example, quaternary ammonium compounds, (g) wetting agents, as for example cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Prevention of the action of microorganisms may be controlled by addition of any of various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.

### A Cathelicidin Peptide Derivative, Inhibits Severe Acute Respiratory Syndrome Coronavirus-2 and its Drifted Variants

To control the covid-19 pandemic, multiple effective vaccines have been developed, and global vaccinations are in progress. However, the virus continues to mutate. Even when full vaccine coverage is achieved, vaccine-resistant mutants may emerge, thus requiring new annual vaccines against drifted variants analogous to influenza. A complimentary solution to this problem could be developing anti-viral drugs that inhibit SARS CoV-2 and its drifted variants. Such therapies would be invaluable in treating immunocompromised people that sometimes do not fully respond to vaccination.

Host defense peptides represent an ancient arm of the innate immune system. The cathelicidin family of peptides were screened from 16 different species for anti-viral activity. A wild boar peptide and a serine variant mutant were identified as agents that inhibit SARS CoV-2. The serine mutant peptide (hereinafter referred to as “pSer” or “Yongshi”) is believed to act as a viral entry inhibitor. Although it is not intended that embodiments of this disclosure be limited by any particular mechanism, it is believed that following the binding of SARS-CoV-2 to its receptor, the spike protein is cleaved, and heptad repeats 1 and 2 multimerize to form the fusion complex that enables the virion to enter the cell. A deep learning-based protein sequence comparison algorithm and molecular modeling indicates that Yongshi acts as mimetics to the heptad repeats of the virus, and thereby disrupts the fusion process. Experimental data confirms the binding of Yongshi to the heptad repeat 1 with a 5-fold higher affinity than heptad repeat 2 of SARS-CoV-2. Yongshi also binds to the heptad repeat 1 of SARS- and MERS-CoV. Most interestingly, it inhibits drifted variants of SARS COV-2 that were tested, including the alpha, beta, gamma, and delta variants of concern.

Cathelicidin peptides have pre-pro peptide structure which permits its synthesis and storage in an inert form until proteolytic processing during degranulation or phagocytosis releases the antimicrobial domain (hereafter referred to as “cathelicidin peptide”). Most cathelicidin peptides are amphipathic and form either α-helical or β-hairpin secondary structures rich in positively charged amino acids. In general, once cleaved into their active, amphipathic form, cathelicidin peptides insert into the outer membrane of a pathogen and cluster to form channels or pores. In the case of viruses, neutralization can be achieved by the “carpet model” of antimicrobial peptide action, where a threshold concentration of peptide causes rapid disintegration of the viral envelope.

LL-37, the only human cathelicidin, inhibits common respiratory pathogens, like influenza A virus (IAV), respiratory syncytial virus (RSV), rhinovirus (HRV), and adenovirus (ADV). Additionally, cathelicidin peptides from other species, including chickens, cows, pigs, sheep, mice, and opossums, also possess inhibitory activity towards human viral pathogens.

The cathelicidin gene is comprised of a signal peptide, a highly conserved cathelin domain followed by a highly diverse cathelicidin peptide domain. The dichotomy of this highly conserved cathelin domain followed by highly diverse cathelicidin peptides allows for the identification of putative cathelicidin peptides even from previously unexplored species if their genomic sequences are known. Putative cathelicidin peptides from 24 diverse species were identified from known carriers of coronaviruses, including bats, pangolins, humans, beluga whales, snakes, wild boar, pigs, frogs, deer, bison, sperm whales, water buffalo, Hawaiian monk seal, Yak, and Zebu cattle. These peptides were tested for their ability to prevent live SARS-CoV-2 from infecting permissive cells in vitro. A single cathelicidin peptide from the wild boar species, Sus scrofa, was identified which possesses significant SARS-CoV-2 inhibitory activity in vitro. Mutational analysis and further screening of this peptide facilitated the identification of a lead-candidate peptide, Yongshi, which effectively discriminated host cells and viral particles while maintaining inhibitory activity across the alpha (B.1.1.7), beta (B.1.3.5.1), kappa (B.1.1.28.1), gamma (B.1.617.1) and delta (B.1.617.2) variants of SARS-CoV-2. Experiments indicate that Yongshi acts as a viral entry inhibitor by interfering with the SARS-CoV-2 spike protein heptad repeat 1 and 2 multimerization.

### A Cathelicidin Peptide of Wild Boar Origin Inhibits SARS-CoV-2

To generate the cathelicidin peptide library, putative cathelicidin genes were identified from the genomes of other species based on homology to the human LL-37 cathelin domain. For each cathelicidin gene, the C-terminal antimicrobial domain was isolated and produced by solid-phase synthesis as a pure peptide.

For the initial screening of the library, the inhibitory potential of each cathelicidin peptide was evaluated at 50 μM by an in vitro focus forming assay or virus infectivity assay on Vero E6 or Vero E6 cells overexpressing human ACE2 receptor or human embryonic kidney (293) cells expressing human ACE2 receptor. Using a 50% inhibitory cutoff, our initial assay identified SARS-CoV-2 inhibitory activity in 9 out of 50 candidates in the cathelicidin peptide library. Each 50% inhibitory candidate was tested for activity at concentrations ranging from 25 μM to 1.56 μM. Most candidates failed to achieve >50% inhibition below 50 μM. Despite this trend, a candidate related to the wild boar PMAP-36 cathelicidin (PMAP-36R) maintained significant activity even when diluted, with a calculated IC₅₀ of 12.03 μM.

### Mutation Analysis of PMAP-36R Identifies Key Residues for SARS-CoV-2 Inhibition

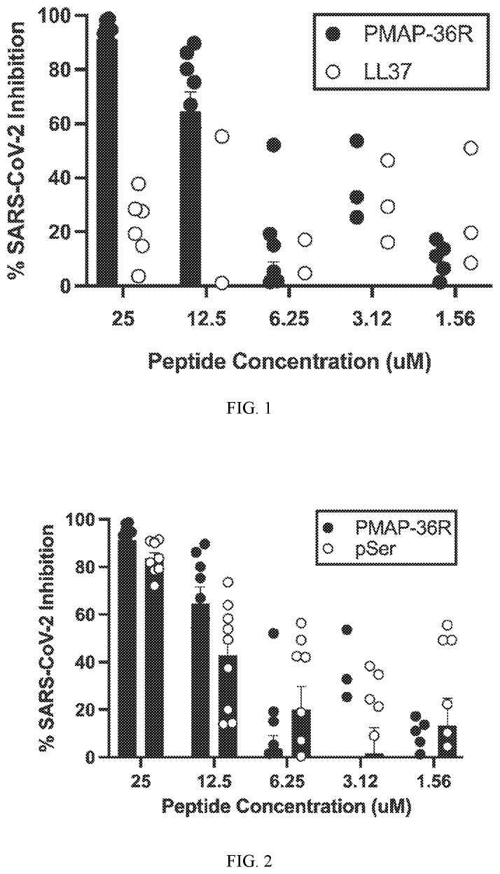

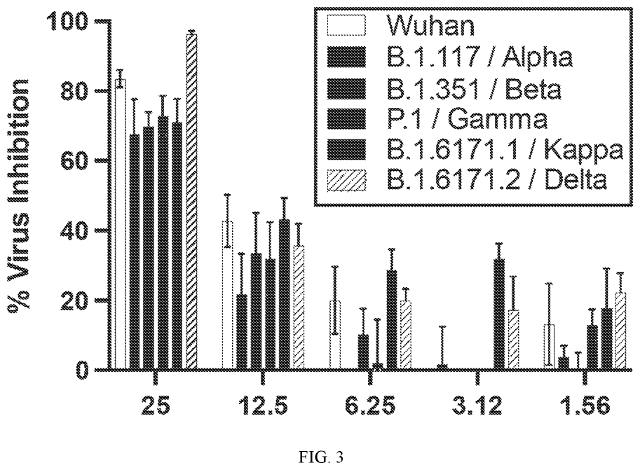

To determine the key residues in PMAP-36R responsible for its high anti-SARS-CoV-2 activity, truncated variants of the parent cathelicidin peptide lacking residues from the N and C termini, as well as variants without the cysteine residues were synthesized and tested. N-terminal truncates lacking 9, 12, 14, or 16 residues (p9N, p12N, p14N, and p16N) displayed a progressive loss of anti-SARS-CoV-2 activity, with no inhibition present in either p14N or p16N. By comparison, the C-terminus was more sensitive to truncation, as loss of the last two residues in p2C immediately impaired function, while little inhibitory activity remained in p5C and none in either p9C or p12C. The activity of these mutants suggested that viral restriction depended on both N and C terminal amino acids. The final two residues of PMAP-36R on the C-terminus include a singular cysteine. Cysteine in a protein is often utilized for producing multimeric protein forms that might be critical for maintaining activity. In order to determine whether cysteine on near the C terminus was important, a mutant of PMAP-36R was made by changing this cysteine to a serine residue. The replacement of C36S in pSer did not reduce SARS-CoV-2 inhibition. The serine mutant form VGRFRRLRKKTRKRLKKIGKVLKWIPPIVGSIPLGSG (SEQ ID NO: 1, Yongshi) inhibited SARS-CoV-2 just as well as the PMAP-36R (FIG. 2).

### The PMAP-36R Serine Mutant (Yongshi) Improves the Viral Specificity by Reducing Cytotoxicity

Host defense peptides, while exhibiting antimicrobial activity, often exhibit toxicity to mammalian cells. To characterize the potential of PMAP-36R for therapeutic applications, its toxicity toward mammalian cells was evaluated in a hemolysis assay with human red blood cells (RBCs). Of the peptides tested, LL-37 and PMAP-36R had significant hemolytic activity. Interestingly, the C36S mutant pSer exhibited significantly reduced hemolytic activity.

Cytotoxicity of peptides were tested using Vero E6 and HEK293T cells to identify general and cell line-specific sensitivities. Cell viability by was quantified a formazan formation assay. As in the hemolysis results, pSer exhibited decreased cytotoxicity compared to the parent PMAP-36R and LL-37. The toxicity from each peptide appeared independent of cell type, producing similar trends in all tested cell lines. In contrast to the hemolysis assay, PMAP-36R has greater toxicity than LL-37 when administered over 48 hours, suggesting that it may act via a slower mechanism than LL37 or differentially target the membranes of RBCs and adherent cell lines.

The therapeutic index (TI) is a measure of safety for therapeutic drugs and, in this context, is the ratio of a compound's IC₅₀ to its TD₅₀. In this case, TI represents the capacity of a cathelicidin peptide to discriminate between SARS-CoV-2 virions and Vero-E6 cell membranes. LL-37 demonstrated no therapeutic potential, with a TI of <1, as neutralization consistently lagged toxicity. The TI was slightly improved for PMAP-36R at 1.94, indicating a modest targeting of SARS-CoV-2 over Vero E6 cells. However, Yongshi displayed heightened specificity for SARS-CoV-2 with a TI of 3.50.

### The Serine Mutant (Yongshi) Requires Chirality for its Virus-Inhibitory Activity

Experiments were performed to determine whether peptide chirality had an impact on the effectiveness of anti-viral activity. The serine mutant (Yongshi) was synthesized using D rather than L amino acids. The resulting peptide, D-Yongshi enantiomer, has a mirrored 3D conformation, permitting the dissection of Yongshi's inhibitory activity into sequence-based and structure-based components. Compared to the wild-type L-Yongshi peptide, D-Yongshi possessed significantly increased cytotoxicity without a comparable increase in SARS-CoV-2 virus inhibition (TI=1.64). The inability of D-Yongshi to inhibit SARS-CoV-2 supports the existence of specific interactions between Yongshi and SARS-CoV-2 dictated by secondary structure.

### The Serine Mutant (Yongshi) Inhibits Emerging SARS-CoV-2 Variants of Concern

Since its emergence in late 2019, SARS-CoV-2 has continued to change: multiple new variants of concern have emerged, defined primarily by acquired mutations in the spike protein. Mutations to the receptor-binding domain, such as, L452R, E484K and N501Y have garnered particular concern as they threaten to erode the protection provided by the original, Wuhan isolate-based vaccines and monoclonal antibody therapies (citations). Since Yongshi was identified by screening peptides against the Wuhan isolate of SARS-CoV-2, experiments were performed to determine the extent to which Yongshi would inhibit drifted variants of SARS-CoV-2. Yongshi was tested against the four currently dominant variants of concern: B.1.1.7 (alpha), B.1.351 (beta), B.1.1.28.1 (gamma), B.1.617.2 (delta), and one newly emergent variant of interest B.1.617.1 (kappa). The defining mutations of each variant are mostly concentrated in the spike 51 region responsible for ACE2 binding. The mutations in the spike protein for these variants are as follows. Alpha (69-70del, 144del, E484K, S494P, N501Y, A570D, D614G, P681H, T716I, S982A, D1118H), Beta (D80A, D215G, 241-243del, K417N, E484K, N501Y, D614G, A701V), Gamma (L18F, T20N, P26S, D138Y, R190S, K417T, E484K, N501Y, D614G, H655Y, T1027I), Kappa (T95I, G142D, E154K, L452R, E484Q, D614G, P681R, Q1071H), and Delta (T19R, V70F, T95I, G142D, E156-, F157-, R158G, A222V, W258L, K417N, L452R, T478K, D614G, P681R, D950N). The spike S2 region responsible for viral fusion is heavily conserved, especially regions corresponding to the fusion peptide and heptad repeats. The fusion peptide is conserved across all 5 variants, and only the delta variant possesses a mutation in a heptad repeat (D950N). Yongshi inhibited all the variants tested. The delta variant which has a mutation in the HR1 region was also inhibited by Yongshi.

1. A method of treating or preventing a viral infection comprising administering an effective amount of a peptide, or a vector encoding a peptide, comprising the amino acid sequence VGRFRRLRKKTRKRLKKIGKVLKWIPPIVGSIPLGSG (SEQ ID NO: 1) or variant thereof.

2. The method of claim 1, wherein the viral infection is a coronavirus infection such as SARS-Cov-2.

3. The method of claim 1, wherein the variant has the amino acid sequence GRFRRLRKKTRKRLKKIGKVLKWIPPIVGSIPLGCG (SEQ ID NO: 2).

4. The method of claim 1, wherein the peptide, or vector encoding a peptide, is administered in combination with another antiviral agent.

5. The method of claim 1, wherein the subject is more than 55 years old.

6. The method of claim 1, wherein the subject is diagnosed with a severe acute infection requiring intensive care.

7. A peptide having the amino acid sequence of VGRFRRLRKKTRKRLKKIGKVLKWIPPIVGSIPLGSG (SEQ ID NO: 1).

8. The peptide of claim 7 having a N-terminal acetyl group.

9. The peptide of claim 7 having a C-terminal amide.

10. The peptide of claim 7 wherein a proline has a hydroxyl substituent.

11. The peptide of claim 7 conjugated to polyethylene glycol.

12. The peptide of claim 7 wherein an amino, carboxyl, or hydroxyl group is substituted with a substituent.

13. A vector comprising a nucleic acid encoding a peptide having the amino acid sequence of VGRFRRLRKKTRKRLKKIGKVLKWIPPIVGSIPLGSG (SEQ ID NO: 1).

14. An expression system comprising a recombinant vector of claim 7.

15. A cell comprising a recombinant vector of claim 7.

16. A pharmaceutical composition comprising a peptide comprising the amino acid sequence of VGRFRRLRKKTRKRLKKIGKVLKWIPPIVGSIPLGSG (SEQ ID NO: 1) and pharmaceutically acceptable excipient.

17. The pharmaceutical composition of claim 16 in the form of a sterilized pH buffered aqueous salt solution.

18. The pharmaceutical composition of claim 16 in the form of a capsule, tablets, pill, powder, or granule.

19. The pharmaceutical composition of claim 16 in the form of a container configured to spray a liquid or a sealed container with a propellant.
